(19) United States
(12) Patent Application Publication
(10) Pub. No.: US 2019/0388122 A1
(43) Pub. Date: Dec. 26, 2019

(54) SYSTEMS AND METHODS FOR CONTACTLESS CRANIO-MAXILLO-FACIAL DISTRACTION

(71) Applicants: THE TRUSTEES OF THE UNIVERSITY OF PENNSYLVANIA, Philadelphia, PA (US); THE CHILDREN'S HOSPITAL OF PHILADELPHIA, Philadelphia, PA (US)

(72) Inventors: Jesse Taylor, Gladwyne, PA (US); Ari M. Wes, Philadelphia, PA (US); Rahul Mangharam, Philadelphia, PA (US); Marco Becanni, Santa Clara, CA (US)

(73) Assignees: THE TRUSTEES OF THE UNIVERSITY OF PENNSYLVANIA, Philadelphia, PA (US); THE CHILDREN'S HOSPITAL OF PHILADELPHIA, Philadelphia, PA (US)

(21) Appl. No.: 16/563,241

(22) Filed: Sep. 6, 2019

Related U.S. Application Data
(63) Continuation of application No. PCT/US2018/021269, filed on Mar. 7, 2018.
(60) Provisional application No. 62/469,178, filed on Mar. 9, 2017.

(51) Int. Cl. A61B 17/66; A61B 17/80

(52) U.S. Cl. CPC A61B 17/663; A61B 2017/00411; A61B 17/8019

(57) ABSTRACT

The presently disclosed subject matter provides systems and methods to enhance fascia closure using surgical mesh products. An apparatus for performing distraction osteogenesis, the apparatus can include a first device that is fully implanted under the skin of a patient. The first device can include a distraction element configured to perform distraction osteogenesis on a bone to which the distraction element is attached and a first magnetic element connected to the distraction element. The apparatus can include a second device that is placed outside the skin of the patient. The second device can include a second magnetic element configured to transfer magnetic torque to the first magnetic element, resulting in the distraction element to perform distraction osteogenesis. The second device can include a processing circuitry configured to control operation of the second magnetic element.

### CROSS-REFERENCE TO RELATED APPLICATIONS

This application is a Continuation of International Patent Application No. PCT/US2018/021269, filed Mar. 7, 2018, which claims priority to U.S. Provisional Application Ser. No. 62/469,178, filed on Mar. 9, 2017, which are incorporated by reference herein in their entirety.

### BACKGROUND

Certain surgical techniques for skeletal expansion include osteotomies, acute movements of variable magnitude, and the necessity for bone grafts. Donor site morbidity, unpredictable resorption of large grafts, and the risk of relapse because of soft tissue resistance to large skeletal movements are some of the several challenges and/or limitations faced by such techniques. Many of these limitations can be avoided by the use of distraction osteogenesis (DO) to lengthen and/or expand the skeleton. Distraction osteogenesis is a technique of bone lengthening which uses the bone's natural healing process. Distraction osteogenesis can be used to gradually expand both bone and soft tissues and minimize bony relapse rates, blood loss, operative time, pen-operative morbidity, and the duration of hospital stays when compared with analogous open, single-staged procedures.

In distraction osteogenesis, a surgeon can attach a device called a distractor to cut bone. Certain devices for craniofacial distraction require a transmucosal or transcutaneous activator. The distractor can be placed under the skin or it can be attached to a skull and face bones on the outside of their skin. Distraction osteogenesis can be a useful tool in the craniofacial surgeon's arsenal, as it can gradually expand both bone and soft tissues, leading to lower bony relapse rates, less blood loss, lower operative time, less pen-operative morbidity, and shorter hospital stays when compared with analogous open, single-staged procedures.

Despite the advantages provided, certain techniques for performing distraction osteogenesis have with a number of limitations. Certain distractors have an external component that protrudes through the skin to allow for manual engagement of the distractor (e.g., a screwdriver) for multiple weeks. Such an external component can predispose the patient to morbidity such as soft-tissue infection, patient and/or parent noncompliance, patient discomfort and/or increased analgesic use throughout the distraction period, as well as unfortunate scarring. In addition to increased morbidity, the manual method by which certain distractors are engaged can eliminate the possibility of using mechanical feedback during the distraction process to aid in diagnosis, and/or prevention of, the most serious drawbacks of distraction (e.g., premature bony consolidation or fibrous non-union of the bony segments, and device failure).

Accordingly, there is a need for an automated technique for performing distraction osteogenesis that incorporates mechanical feedback during the distraction process. Additionally, there remains a need for fully implantable distractor that does not protrude through the skin and can be controlled by components of the system completely outside the patient.

### BRIEF DESCRIPTION OF THE DRAWINGS

FIGS. 1A and 1B illustrate an exemplary system for performing distraction osteogenesis in accordance with the present disclosure. FIG. 1A is a diagram illustrating the placement of the disclosed distraction osteogenesis system on a human skull. FIG. 1B is a block diagram illustrating a system level diagram of the disclosed distraction osteogenesis system.

FIGS. 2A and 2B illustrate an exemplary distraction device used in the disclosed system for performing distraction osteogenesis in accordance with the present disclosure.

FIG. 3 is a diagram illustrating a finite state machine model used by the disclosed subject matter to perform distraction osteogenesis in accordance with the present disclosure.

FIG. 4 is a diagram illustrating a Hall Effect sensor that can be used with and/or as a part of the disclosed distraction osteogenesis system in accordance with the present disclosure.

FIG. 5 illustrates a Hall Effect sensor used with a magnet in the disclosed system in accordance with the present disclosure.

FIGS. 6A and 6B are screenshots illustrating different views of an exemplary prototype of the disclosed system for performing distraction osteogenesis in accordance with the present disclosure.

Throughout the figures, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments.

### DETAILED DESCRIPTION

The subject matter disclosed herein provides systems and methods for performing distraction osteogenesis. Distraction osteogenesis is the process by which new bone is generated at an osteotomy site by gradually separating two opposing bony fragments.

In some embodiments, the bones of the human skull can be joined by cranial sutures. The cranial sutures can gradually fuse within the first few years after birth in infants. In the event that one or more of the sutures fuse too early, the growth of the skull can become restricted, resulting in limited space for the brain and can result in abnormal head shape. In order to stop such unexpected early fusing, cranial distractors can be used to pull the skull gradually (e.g., approximately 1 mm each day). Parents of an infant patient can use a patient screwdriver to rotate the rod of distracters to distract the skull. By using the disclosed system for performing distraction osteogenesis, the required daily amount of distraction can involve two turns of 360° for a total of 1 mm of distraction each day.

In some embodiments, new bone can be generated, using the distraction osteogenesis process, at an osteotomy site by gradually separating two opposing bony fragments. In particular, an osteotomy can be made through the bone which will be expanded, and a plate can be then attached to each of the resulting bone fragments, with a driveshaft connecting the two aforementioned plates. Activation of the distraction device involves displacement of the two bone-attached plates relative to one another, and can occur through the application of torque to the driveshaft. This process of activation can occur, e.g., one to three times per day, in order to achieve a daily distraction rate of the surgeon's choosing (e.g., 1-2 mm per day). The number of days for which distraction takes place can depend on the specific deformity of the patient, and the distraction length can vary.

In some embodiments, the disclosed system can include a magnetic-driven motorized cranial vault distractor embedded system. In some embodiments, the magnets and distractors used in the disclosed system can be enclosed completely under the skin to reduce infection. The disclosed system can include an external band that can be worn on the patient's head. The external band can include a stepper motor, microprocessor and/or microcontroller, and a sensor that can be used to control the rod rotation with precision. The disclosed system can include a fast rotation mode and a slow rotation mode, which can complete the rotation over a period of time to reduce the baby's pain. In some embodiments, the internal distractor can be controlled using Bluetooth.

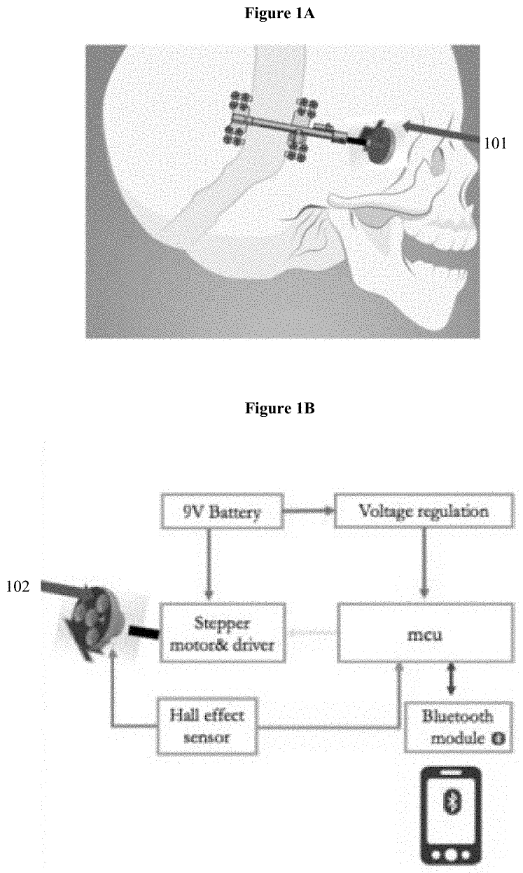

FIG. 1A is a diagram illustrating the placement of the disclosed distraction osteogenesis system on a human skull. As shown in FIG. 1A, the disclosed system can include at least two different sub-systems: an internal subsystem that is located under the skin of the patient and an external subsystem that is located outside the patient (e.g., worn on a headband on the patient's skull).

In some embodiments, the internal subsystem can include a distractor and a disk 101 located at the end of the distractor rod and can have magnets mounted on it. The internal subsystem can be a fully implantable distractor with a magnetic component that can be affixed to the activation site. The activation site of the distractor can be the most proximal location where applied torque to a driveshaft can cause linear displacement of the two bony-fixation-plates.

In some embodiments, the external subsystem can include a headband that can pair magnetically with the magnetic component of the internal subsystem's distractor. The headband can transmit torque to the internal subsystem, causing the distractor to perform distraction. In some embodiments, the external subsystem can be controlled through a Bluetooth or Wi-Fi enabled device, and can be confined to a preset, surgeon specified distraction protocol. The external subsystem can include a stepper motor to create torque to spin a disk with magnets to transfer torque to the internal subsystem. The external subsystem's disk can be positioned such that it located on the head at a position that is aligned with the disk of magnets of the internal subsystem. The external subsystem's disc of magnet(s) can apply and/or transfer magnetic torque to the disc of magnets in the internal subsystem. Upon application of magnetic torque on the internal subsystem's disc of magnet(s) by the external subsystem's disc of magnet(s), the internal subsystem can rotate and accordingly cause the distractor rod connected to the internal subsystem's disc of magnet(s) to rotate as well, resulting in distraction being performed as a result of the rotation.

In some embodiments, the user and/or parent can use the disclosed system to perform a specific amount of distraction each day. For example, for a predetermined amount of time (e.g., two to three weeks) after surgery while the child patient is at home, a parent or caregiver can perform distraction of about 1 to 2 millimeters a day. Such a predetermined amount of controlled distraction can maintain tension on the wires and can move the facial bones apart. As a result of such controlled distraction, new bone can grow to fill in the gaps left by the distraction and can harden over time. Once the bones are in the right position, the distraction can be stopped and the new bones generated due to the distraction can heal in their new positions, which is often known as consolidation, or the “healing phase” and can last one to two months.

FIG. 1B is a block diagram illustrating a system level diagram of the disclosed distraction osteogenesis system. In particular, the block diagram of FIG. 1B illustrates a block diagram of the external subsystem. As shown in FIG. 1B, the external subsystem can further include a microprocessor, a power supply, a stepper motor and driver 102, wireless communication module (e.g., Bluetooth module) for another device (e.g., mobile device) to communicate and control the disclosed system, and a sensor for measuring rotations of both the magnetic components of the internal subsystem and the external subsystem (e.g., Hall Effect sensor). The external subsystem can further include a power supply with power conversion and management module (e.g., voltage regulation module) to provide stable and clean power to the internal subsystem.

In some embodiments, the disclosed system can be configured to provide adequate torque with high accuracy (e.g., to the sub-Newton-meter and/or Newton-millimeter scale). For example, when the external subsystem and the internal subsystem are paired together using magnetic components instead of a direct connection, an adequate amount of torque can be needed by the internal subsystem to be supplied from the detached arm in order to distract bones. The amount of torque needed can be greater than those applied by the bones. Enough torque and indication signals need to be transferred from the external subsystem to the internal subsystem to satisfy the amount of torque required by internal subsystem to perform the distraction. The amount of magnetic force needed to generate this amount of torque can be calculated to meet these requirements of the internal subsystem. In some embodiments, the distribution of magnets on both the internal and external subsystem can be carefully designed not to draw too much attraction force between the magnets in the internal and external subsystem to prevent skin damage.

In some embodiments, the magnet(s) on the internal subsystem and the external subsystem can be paired when they are brought in close proximity of each other and a magnetic field is enabled. For example, the internal subsystem can be placed within millimeters to a centimeter of the skin under the soft tissue to be close to the skin and the external subsystem can be placed on the outside of the skin to be touching the skin to be as close to the internal device as possible. For example, the disc of magnet(s) of the external subsystem can move in a characteristic manner (e.g., perform one or more partially clockwise rotations for a predetermined amount of degrees and then rotate counterclockwise for another predetermined amount of degrees and/or vice versa) to generate a similar movement on the disc of magnet(s) in the internal subsystem without performing any distraction in order to determine if both the internal and external subsystems are aligned and located in the appropriate locations to enable magnetic coupling such that the external subsystem can transfer magnetic torque to the internal subsystem. The Hall Effect sensor can determine how the disc of magnet(s) on the internal subsystem responds to the movement of the disc of magnet(s) of the external subsystem. If the microprocessor determines from the Hall Effect sensor measurements that the internal subsystem's disc of magnet(s) responds with the same characteristic movement produced by the external subsystem's disc of magnet(s), the processor can determine that the external subsystem and the internal subsystem are paired. The microprocessor of the external subsystem can instruct a visual indicator on the external subsystem and/or an electronic device (e.g., a mobile device) connected through the Bluetooth module to the external subsystem to display an indicator and/or a message indicating the external and internal subsystem have been paired and are ready to commence the distraction process. In some embodiments, the external subsystem's magnets and the internal subsystem's magnets can be interdigitated such that these magnets are not attracted to each other but that instead the center of the internal and external subsystems are attracted to each other.

In some embodiments, once the microprocessor of the external subsystem determines that the external subsystem's disc of magnet(s) has been paired with the internal subsystem's disc of magnet(s), the microprocessor can instruct the external subsystem's disc of magnet(s) to apply a predetermined amount of magnetic torque on the internal subsystem's disc of magnet(s) for a predetermined duration of time in order to perform the amount of distraction at a given time according to parameters prescribed by the surgeon and/or the parent.

In some embodiments, the microprocessor in the external subsystem can instruct the stepper motor to generate enough magnetic torque such that the magnets in the internal subsystem can move the bony footplates away from each other in a particular direction (e.g., along a first axis) by the predetermined amount needed per day. For example, the stepper motor can provide the magnetic torque necessary such that the magnets in the internal subsystem can spatially separate and/or move the first bony footplate relative to the second bony footplate in the internal subsystem's distractor. In some embodiments, the microprocessor can be configured to perform the distraction using customizable variables (e.g., linear rate, constant force, variable force, rapid alternating forces). For example, the microprocessor can instruct the stepper motor to generate enough magnetic torque such that the magnets in the internal subsystem can move one or more of the bony footplates at a linear rate, at a constant force, or with rapid alternative movements (e.g., vibrating adjacent to one another) to perform distraction osteogenesis.

In some embodiments, the microprocessor can receive initial distraction parameters and parameters for incremental distraction from an external control device (e.g., mobile device of a surgeon and/or patient's parent) wirelessly in communication with the external subsystem. For example, the external control device can provide an initial amount of distraction length that the first bony plate should move with respect to the second bony plate. Upon receiving such distraction parameters, the processing circuitry of the external subsystem can calculate the amount of magnetic torque that needs to be generated and transferred to the magnets on the internal subsystem such that the distractor arm can separate bony footplates by the amount of distance specified by the external control device.

In some embodiments, the disclosed system can support two modes of operation in the disclosed system for performing the distraction: a fast rotation mode and a slow rotation mode. Under the fast rotation mode, the microprocessor can instruct the external subsystem's disc of magnet(s) to apply a magnetic torque on the internal subsystem's disc of magnet(s) such that the internal subsystem's disc of magnet(s) can rotate at a speed that allows distraction of 1 mm to be performed in less than 1 minute. Under the slow rotation mode, the microprocessor can allow the user to specify the time period over which the user desires to have the internal subsystem's disc of magnet(s) perform one 360° rotation. The user can transmit such parameters for the slow rotation mode from the Bluetooth connected device (e.g., mobile device) to the microprocessor of the external subsystem. Upon receiving such a control parameter, the microprocessor can calculate the amount of magnetic torque that the external subsystem's disc of magnet(s) should apply on the internal subsystem's disc of magnet(s) to have the internal subsystem's disc of magnet(s) and/or the distractor rod coupled to the internal subsystem's disc of magnet(s) to perform a single 360° rotation over the user specified duration of time. Under the slow rotation mode, the distraction rate can be user controlled. For example, if the patient experiences pain and/or discomfort due to the distraction performed under the fast rotation mode, the user can set a slower rotation speed that does not cause such discomfort using the slow rotation mode.

In some embodiments, the stepper motor can be used for precise rotation control. The stepper motor can be an open loop device. To ensure the system operates in a stable and precise manner, the loop can be closed by placing a Hall Effect sensor on the external subsystem to detect the rotation of the magnets in both the external and internal subsystems. The Hall Effect sensor can act as a wheel encoder to count the number of degrees that the disc of magnets on both the external and internal subsystems have rotated. By determining the number and type of magnets (e.g., previously set in the microprocessor of the external device and/or can be manually altered by having a user specify the number and types of magnets that are in the internal and/or external disc of magnet(s)) that are placed on both the internal and external subsystems' disc of magnet(s) and a voltage measurement received from the Hall Effect sensor, the microprocessor can determine the total amount of rotation performed by each of the external and internal subsystems' respective disc of magnet(s). The Hall Effect sensor can determine how many rotations are performed by the internal subsystem's disc of magnet(s) in response to a rotation performed by the external subsystem's disc of magnet(s) to maintain a ratio. The Hall effect sensor can transmit such data to the microprocessor, which can then adjust the magnitude of the magnetic field to be applied by the external subsystem's disc of magnet(s) on the internal subsystem's disc of magnet(s) in order to achieve a desired ratio of measured rotations between the internal subsystem's disc of magnet(s) to that of the external subsystem's disc of magnet(s).

In some embodiments, the Bluetooth module communicating with the microprocessor can provide an interface with any device with a standard Bluetooth module. A user can use their Bluetooth connected device (e.g., mobile device) for receiving commands and sending rotation information to the external subsystem. The external subsystem can include multiple (e.g., five) light emitting diodes (LEDs) to indicate the progress of rotation.

In some embodiments, the external subsystem can be configured to transmit relevant data to a surgeon and/or a device used by the surgeon. For example, the external subsystem can transmit measurement results of the distraction that it can calculate using the Hall Effect sensor's measurement of the amount of rotation detected in the internal subsystem's magnet(s) to a surgeon's mobile device. The external subsystem can also receive control parameters from the surgeon's device, which the external device can use to determine how to adjust the amount of magnetic torque that is transferred from the magnet(s) of the external subsystem to the magnet(s) of the internal subsystem.

In some embodiments, the external subsystem can include a visual indicator that indicates different operations statuses and/or alarms based on measurement data received from the internal subsystem's magnet from the Hall Effect sensor. For example, the visual indicator can indicate whether successful pairing between the internal subsystem and the external subsystem was achieved, whether additional distraction is required, and whether the internal subsystem's magnet is being rotated at speeds and/or quantities above that of a predetermined threshold, causing the bones to be distracted too fast (e.g., an alarm condition). Based on measurement data received from the Hall Effect sensor's measurements of the magnet(s) on the internal subsystem and the external subsystem, the microprocessor can determine the presence of these different conditions and/or statuses of the distraction performed by the internal subsystem and accordingly can indicate such information on the visual indicator.

In some embodiments, the disclosed subject matter can provide an indication that the specified distraction parameters sent to the internal subsystem (e.g., by way of magnetic torque) falls outside a predetermined operating range of forces for the distraction parameters. For example, the microprocessor can determine that the forces required to perform distraction according to the parameters specified by the user device, communicatively coupled to the external subsystem through the Bluetooth and/or wireless module, fall outside a predetermined operating range of distraction forces. Additionally or alternatively, the external subsystem can determine, from the distraction parameters that are inputted, that the specified distraction parameters fall outside the predetermined operating range.

In some embodiments, when the distraction parameters fall outside the operating range, the internal subsystem can still proceed with performing the distraction while the external subsystem displays a warning message and/or turns on a warning indicator. For example, the external subsystem can log and/or record that the force required to perform the distraction falls outside the predetermined operating range and transmit such information to the user device while performing the distraction.

In some other embodiments, when the distraction parameters fall outside the operating range, the external subsystem can stop the distraction from occurring and instruct a warning indicator on the external subsystem and/or a display on the Bluetooth connected user device to display a warning message, providing an override to the distraction if the distraction parameters fall outside the operating range. Once a user responds to the warning message on the user device and allows the distraction to occur despite the distraction parameters falling outside the predetermined operating range, the external subsystem can proceed with performing the distraction. Additionally or alternatively, the user can be provided with an option to modify the distraction parameters once it is determined that the distraction parameters fall outside the predetermined operating range. In some embodiments, the microprocessor can determine that a torque transferred to the distraction device by the external subsystem's disc of magnet(s) is outside (e.g., exceeds or falls below) a predetermined operating range of torque for the distraction device. In response to determining that the torque transferred to the distraction element of the first device by the second device is outside the predetermined operating range of torque for the distraction element, the microprocessor can instruct the external subsystem's disc of magnet(s) to adjust (e.g., increase or decrease) a rate of rotation. In some embodiments, the microprocessor can measure a distance that the distraction device has distracted. The microprocessor can stop the distraction device from performing further distraction if the measured distance exceeds a preset distraction distance.

In some embodiments, the microprocessor can instruct a display (e.g., located on the external subsystem and/or a display on the wirelessly connected user device) to display which direction the external subsystem should move, during docking, for optimal placement of the second device with respect to the first device for the first and second device to be paired. For example, the Hall Effect sensor can measure the distance and direction of movement of the external subsystem's disc with respect to the internal subsystem's disc during docking and/or initial placement of the external subsystem on the patient's skull. The processor, using such information from the Hall Effect sensor, can display on a display device which direction the external subsystem needs to move for optimal placement on the patient's skull during docking in order for the external subsystem to be paired with the internal subsystem.

In some embodiments, user of the disclosed system can provide significant advantages over certain conventional distraction devices. For example, use of an automated and controlled distraction process in which the microprocessor can control the rotations of the internal subsystem can decrease errors performed in manual distraction (e.g., manual turning of screws for distraction) and can result in a less traumatic experience for patients. The disclosed system can also automatically upload information and metrics of the distraction as measured by the Hall Effect sensor and analyzed by the microprocessor to an external device using the wireless and/or Bluetooth module. Additionally, the disclosed system provides for an easy method to control the distraction rate by using the slow rotation mode. Additionally, the use of a completely internal subsystem and an external subsystem that are coupled magnetically without any components protruding through the skin and/or wound significantly decreases the risk of infection and/or other complications.

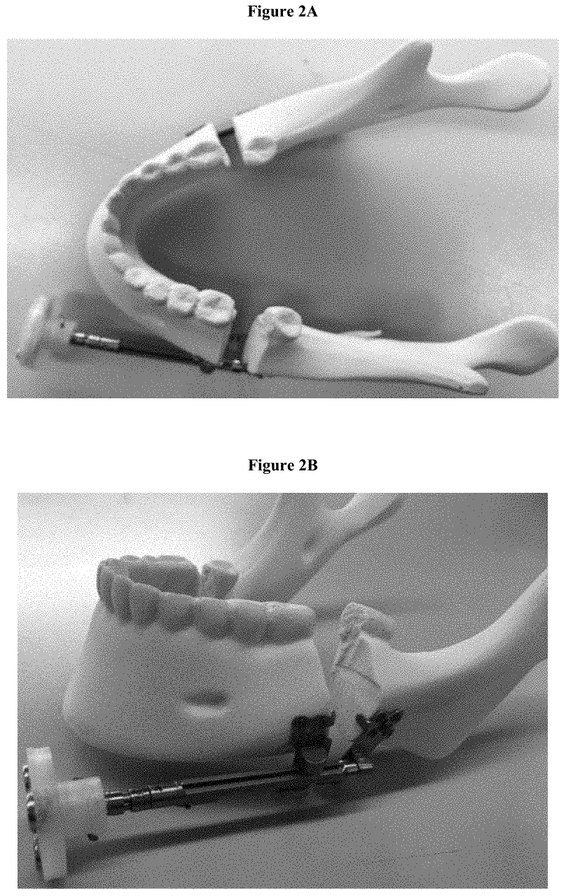

FIGS. 2A and 2B illustrate an exemplary distraction device (e.g., the internal subsystem) used in the disclosed system for performing distraction osteogenesis. FIG. 2A illustrates a top-down view of an exemplary distractor of the disclosed system's internal subsystem that is attached to a portion of a patient jawbone to perform distraction osteogenesis on the patient's broken jawbone. FIG. 2B illustrates a side view of the exemplary distractor that is attached to a portion of a patient jawbone to perform distraction osteogenesis on the patient's broken jawbone. As illustrated in FIGS. 2A and 2B, the distractor's bony plates can be attached to the different portions of the broken jawbone. Upon receiving a magnetic torque from the external subsystem, the disk attached to the distractor rod of the internal subsystem shown in FIGS. 2A and 2B can bring the two bony plates together by a distance proportionate to the amount of magnetic torque received from the external subsystem. The internal subsystem can be completely implanted under the patient's skin and be internal to the patient without protruding externally.

In some embodiments, the distractor arm can be telescoping and/or collapsible arm that is coupled to a first bony footplate and a second bony footplate. In some embodiments, the second bony footplate can stay fixed while the distractor arm can move the first bony footplate as instructed by the processing circuitry.

In some embodiments, small magnets, and/or various different distributions of magnets on the disk in the internal subsystem can be used. In some embodiments, the magnet(s) used in the internal device can be weaker in magnetic strength compared to the magnet(s) used in the external device to ensure that the patient's safety is not comprised due to strong magnetic forces resulting from the internal subsystem's magnets placed within the patient's body. In some embodiments, the external subsystem's circuitry can be implemented on a printed circuit board or an integrated circuit for a compact design. In some embodiments, the a micro-stepper motor and/or other types of motors with closed-loop functionalities can be utilized for controlling the magnetic torque to be generate for transfer to the internal subsystem.

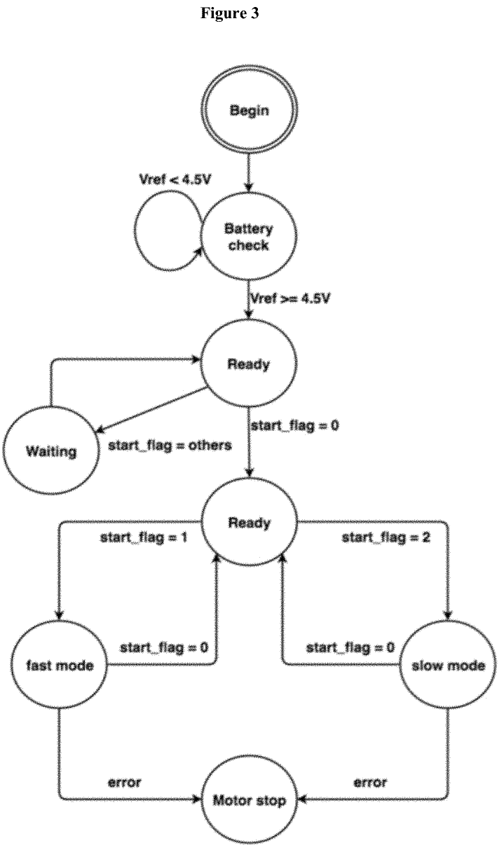

FIG. 3 is a diagram illustrating a finite state machine model used by the disclosed subject matter to perform distraction osteogenesis. In the embodiment illustrated in FIG. 3, upon initiation, the system can enter a state to check whether there adequate power supplied to the system. For example, the microprocessor can determine whether the reference voltage of 4.5V is being provided by the battery module. The microprocessor can remain in this state until it determines that the battery pack is indeed supplying the required reference voltage (e.g., 4.5V) before progressing to a ready state. As shown in the embodiment illustrated in FIG. 3, if the external device receives an incorrect and/or invalid selection (e.g., start_flag=others), the microprocessor remains in the ready state and does not proceed to change state. If the external device does not receive any additional input, determines that the stop and/or pause button on the external device and/or user device paired to the external system wirelessly has been activated and/or that the external device has been turned off (e.g., start_flag=0), the system remains in the ready state. If the external system and/or the microprocessor determines that a selection has been made to initiate the fast rotation mode (e.g., start_flag=1), the microprocessor can instruct the disc of magnet(s) on the external device to rotate according to the predetermined speed and/or duration that has been preset to correspond to the fast rotation mode (e.g., for the internal subsystem's disc of magnet(s) to rotate at a speed that allows distraction of 1 mm to be performed in under 1 minute). If the external system and/or the microprocessor determines that a selection has been made to initiate the slow rotation mode (e.g., start_flag=2), the microprocessor can instruct the disc of magnet(s) on the external device to rotate according to a calculated speed that would correspond to the user specified distraction rate. The microprocessor can track which state the system is in. If the microprocessor determines that it does not receive any additional input after rotation has stopped, and/or determines that the stop and/or pause button on the external device and/or user device paired to the external system wirelessly has been activated and/or that the external device has been turned off, the microprocessor can instruct the external subsystem's disc of magnet(s) to stop rotating and for the system to switch to the ready state. If the microprocessor determines, using the Hall Effect measurements and/or any other sensors and/or input on the external device, that an error condition (e.g., a high surge detected by the Hall Effect sensor) has occurred while the system is in the fast rotation mode or the slow rotation mode, the microprocessor can instruct the stepper motor to stop and/or the external subsystem's disc of magnet(s) to stop rotating.

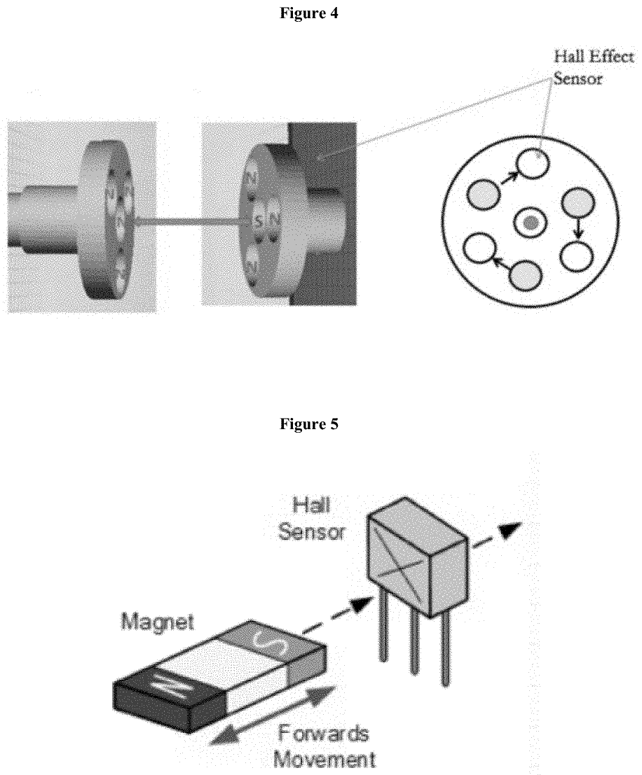

FIG. 4 is a diagram illustrating a disc of magnets that can be used with and/or as a part of the disclosed distraction osteogenesis system. Each disc can include a central magnet that is located at the center of the disc and at least one perimeter magnets that are located along the perimeter of the disc.

In some embodiments, the central magnet on the external subsystem's disc can be of an opposite polarity than the central magnet on the internal subsystem's disc so that the internal subsystem and external subsystem's discs are attracted to each other at the center. The perimeter magnet(s) on the internal subsystem's disc can be interdigitated with the perimeter magnet(s) on the external subsystem's disc. The perimeter magnet(s) on the internal subsystem's disc can have the same polarities with respect to the perimeter magnet(s) on the external subsystem's disc. Accordingly, when the external subsystem's disc rotates the perimeter magnets on the external subsystem's disc repel the perimeter magnets on the internal subsystem's disc while the centers of the internal and external subsystems' discs are attracted to each other, causing the internal disk to rotate in the same manner as the external disc.

In some embodiments, the central magnet on the external subsystem's disc can be of the same polarity than the central magnet on the internal subsystem's disc so that the internal subsystem and external subsystem's discs repel each other at the center. The perimeter magnet(s) on the internal subsystem's disc can be aligned with the perimeter magnet(s) on the external subsystem's disc. The perimeter magnet(s) on the internal subsystem's disc can have different polarities with respect to the perimeter magnet(s) on the external subsystem's disc. Accordingly, when the external subsystem's disc rotates the perimeter magnets on the external subsystem's disc attract the perimeter magnets on the internal subsystem's disc while the centers of the internal and external subsystems' discs are repelling each other, causing the internal disk to rotate in the same manner as the external disc.

In some embodiments, the external and internal subsystems' discs can use magnetic torque couplers in order to transfer magnetic torque from the external subsystem's disc to the internal subsystem's disc to have the internal disc rotate as desired in response to the instructions of the microprocessor.

FIG. 5 illustrates a Hall Effect sensor used with a magnet in the disclosed system. The Hall Effect sensor can detect the distance of the magnet from the sensor. Using groups of sensors, the Hall Effect sensor can determine the relative position of the magnet that it is measuring. The Hall Effect sensor can measure the magnetic field generated by the magnets on the internal subsystem and the external subsystem's disc of magnet(s). The Hall Effect sensor can vary its output voltage in response to the magnitude of the measured magnetic field. Such voltage differences can be transmitted to the microprocessor of the external subsystem to indicate the measured magnitude of the magnetic field between the internal and external subsystem.

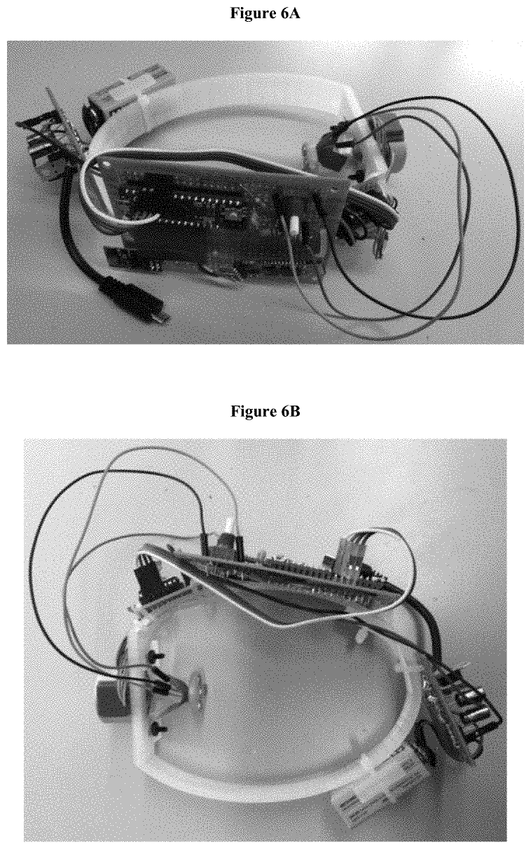

FIGS. 6A and 6B are screenshots illustrating different views of the external subsystem of an exemplary prototype of the disclosed system for performing distraction osteogenesis. The microprocessor, stepper motor, power source, and disc of magnet(s) on the external subsystem can be electrically connected to each other using a breadboard (as shown in FIGS. 6A and 6B) and/or printed circuit board and the combined circuitry and electrical components can be attached to a headband so that the external subsystem can be worn around the skull and placed as close to the internal subsystem as possible on the outside of the patient's skin in a non-invasive manner. In some embodiments, the external subsystem can include an input device for the user to set input parameters including at least one or more of a torque range for the distraction element, an emergency stop command, duration of distraction, and distraction rate. In some embodiments, the one or more magnets on the external subsystem's disc of magnets can be removable from the disc such that a user to replace one or more magnets placed on the disc with different magnets.

It will be understood that the foregoing is only illustrative of the principles of the present disclosure, and that various modifications can be made by those skilled in the art without departing from the scope and spirit of the present disclosure.

1. An apparatus for performing distraction osteogenesis, the apparatus comprising:
a first device that is fully implanted under the skin of a patient, wherein the first device comprises:
a distraction element configured to perform distraction osteogenesis on a bone to which the distraction element is attached;
a first magnetic element connected to the distraction element; and
a second device that is placed outside the skin of the patient, wherein the second device comprises:
a second magnetic element configured to transfer magnetic torque to the first magnetic element, resulting in the distraction element to perform distraction osteogenesis;
a processing circuitry configured to control operation of the second magnetic element.

2. The apparatus of claim 1, wherein the first magnetic element is configured to be magnetically coupled to the second magnetic element, and wherein the first magnetic element is configured to rotate in response to rotation of the second magnetic element as magnetic torque is transferred from the second magnetic element to the first magnetic element as a result of the rotation of the second magnetic element.

3. The apparatus of claim 1, wherein the distraction device comprises a first plate connected to a first portion of the bone and a second portion of the bone, and wherein rotation of the first magnetic element can cause the distraction device to perform distraction osteogenesis by increasing distance between the first plate and the second plate.

4. The apparatus of claim 1, wherein the second device further comprises a wireless communication circuitry that is configured to enable wireless communication between the second device and a user device, wherein the processing circuitry is configured to:
receive distraction parameters from the user device through the wireless communication circuitry; and
transmit measurement data indicating information about the distraction performed by the first device to the user device through the wireless communication circuitry.

5. The apparatus of claim 1, wherein the processing circuitry is configured to adjust a rate of distraction at which the distraction element is to perform distraction osteogenesis by adjusting a rotation rate of the second element.

6. The apparatus of claim 1 further comprising a Hall Effect sensor that is communicatively coupled to the processing circuitry, and wherein the Hall Effect sensor is configured to measure a direction and amount of movement of the first magnetic element and a direction and amount of movement of the second magnetic element; and
wherein the processing circuitry is configured to determine, from measurement information received from the Hall Effect sensor, whether the first magnetic element and second magnetic element are magnetically paired.

7. The apparatus of claim 6, wherein the processing circuitry is configured to adjust a rotation rate of the second magnetic element based on measurement information indicating the direction and amount of movement of the first magnetic element to have the distraction element perform distraction on the bone at a preset rate of distraction.

8. The apparatus of claim 1, wherein the processing circuitry is configured to determine that a torque transferred to the distraction element of the first device by the second device is outside a predetermined operating range of torque for the distraction element.

9. The apparatus of claim 8, wherein in response to determining that the torque transferred to the distraction element of the first device by the second device is outside the predetermined operating range of torque for the distraction element, the processing circuitry instructs the second device to adjust a rate of rotation of the second magnetic element.

10. The apparatus of claim 1, wherein the processing circuitry is configured to measure a distance that the distraction element has distracted and wherein the processing circuitry is configured to stop the distraction element from performing further distraction if the measured distance exceeds a preset distraction distance.

11. The apparatus of claim 1, wherein the processing circuitry instructs a display to display which direction the second device should move, during docking of the second device, for optimal placement of the second device with respect to the first device for the first and second device to be paired.

12. The apparatus of claim 1, wherein the second device comprises an input device for the user to set input parameters including at least one or more of a torque range for the distraction element, an emergency stop command, duration of distraction, and distraction rate.

13. The apparatus of claim 1, wherein the second magnetic element includes one or more removable magnets, and wherein the second magnetic element is configured for a user to replace one or more magnets placed on the second element with different magnets.
